(19) United States
(12) Patent Application Publication
(10) Pub. No.: US 2026/0144496 A1
(43) Pub. Date: May 28, 2026

(54) TERMINAL DEVICE

(71) Applicant: OMRON HEALTHCARE Co., Ltd., Kyoto (JP)

(72) Inventor: Yukiya SAWANOI, Kyoto (JP)

(21) Appl. No.: 19/453,605

(22) Filed: Jan. 20, 2026

Related U.S. Application Data
(63) Continuation of application No. PCT/JP2024/044015, filed on Dec. 12, 2024.

(30) Foreign Application Priority Data
Mar. 5, 2024 (JP) 2024-032857

(51) Int. Cl. A61B 5/00; A61B 5/022; A61B 5/16; G16H 40/67

(52) U.S. Cl. CPC A61B 5/7435; A61B 5/022; A61B 5/165; A61B 5/748; G16H 40/67; A61B 2560/0252

(57) ABSTRACT

A terminal device includes a blood pressure information acquisition unit configured to acquire blood pressure information on a subject measured using a blood pressure monitor, a factor information acquisition unit configured to acquire a plurality of pieces of factor information that affect a blood pressure fluctuation of the subject, an input unit configured to receive an input of a designated unit of period designated from among a plurality of units of period, a selection unit configured to select, from among the plurality of pieces of factor information, first factor information associated with the designated unit of period, and a display control unit configured to display the blood pressure information and the first factor information on a display screen along a time axis based on the designated unit of period.

### CROSS-REFERENCE TO RELATED APPLICATIONS

This application is the U.S. national stage application filed pursuant to 35 U.S.C. 365(c) and 120 as a continuation of International Patent Application No. PCT/JP 2024/044015, filed Dec. 12, 2024, which application claims priority to Japanese Patent Application No. 2024-032857, filed Mar. 5, 2024, which applications are incorporated herein by reference in their entireties.

### TECHNICAL FIELD

### The Present Disclosure Relates to a Terminal Device.

### BACKGROUND ART

In the related art, it has been desirable to repeatedly measure blood pressure values for health management. The measured blood pressure values are used, for example, as a trend graph of blood pressure values to check the tendency of change. An automatic blood pressure measuring device disclosed in JP H10-286241 A (Patent Document 1) is configured to output a trend graph that represents lifestyle-related information and blood pressure values along a predetermined time axis.

### CITATION LIST

### Patent Literature

Patent Document 1: JP H10-286241 A

### SUMMARY OF INVENTION

### Technical Problem

In Patent Document 1, the above-mentioned configuration is used to study the recognition of the relationship between changes in lifestyle-related information such as the amount of exercise and weight and blood pressure values. Factors that affect fluctuations in blood pressure values include, for example, not only the weight and amount of exercise mentioned above, but also various other factors such as room temperature, outside air temperature, sleep, stress, and medication. However, not all of these factors affect short-term (e.g., one day, one week) blood pressure fluctuations, and many factors also affect long-term (e.g., one month, one year) blood pressure fluctuations. Therefore, for example, if short-term blood pressure fluctuations are displayed together with factors that affect long-term blood pressure fluctuations (e.g., outside air temperature, weight), there is a likelihood that the relevance between blood pressure fluctuations and those factors may be misunderstood.

An object of one aspect of the present disclosure is to provide a terminal device that can improve user convenience by presenting a relevance between blood pressure fluctuations during a designated period and factors that affect blood pressure fluctuations during that period.

### Solution to Problem

In an example of the present disclosure, a terminal device includes: a blood pressure information acquisition unit configured to acquire blood pressure information on a subject measured using a blood pressure monitor; a factor information acquisition unit configured to acquire a plurality of pieces of factor information that affect a blood pressure fluctuation of the subject; an input unit configured to receive an input of a designated unit of period designated from among a plurality of units of period; a selection unit configured to select, from among the plurality of pieces of factor information, first factor information associated with the designated unit of period; and a display control unit configured to display the blood pressure information and the first factor information on a display screen along a time axis based on the designated unit of period.

According to the above configuration, the user convenience can be improved by presenting the relevance between blood pressure fluctuations during a designated period and factors that affect blood pressure fluctuations during that period.

In another example of the present disclosure, the selection unit selects second factor information that is not associated with the designated unit of period. The display control unit displays the blood pressure information, the first factor information, and the second factor information on the display screen along a time axis based on the designated unit of period. Each of the blood pressure information and the first factor information is displayed with priority over the second factor information.

According to the above configuration, the user can easily check the first factor information that requires attention, and can also check the second factor information as reference information.

In another example of the present disclosure, the display control unit displays each of the blood pressure information and the first factor information with priority over the second factor information by displaying the second factor information in a grayed-out manner.

According to the above configuration, the user can clearly distinguish between the first factor information and the second factor information.

In another example of the present disclosure, the display control unit controls a display order of the blood pressure information, the first factor information, and the second factor information such that each of the blood pressure information and the first factor information is displayed with priority over the second factor information.

According to the above configuration, the user can clearly distinguish between the first factor information and the second factor information.

In another example of the present disclosure, the plurality of pieces of factor information include a room temperature and an outside air temperature at the time of blood pressure measurement, and a weight, exercise information, sleep information, stress information, medication information, smoking information, and salt intake of the subject.

According to the above configuration, the user can ascertain specific examples of a plurality of pieces of factor information that affect the blood pressure fluctuation of the subject.

In another example of the present disclosure, the plurality of units of period include a unit of a year, a unit of a month, a unit of a week, and a unit of a day. When the designated unit of period is the unit of a year or the unit of a month, the selection unit selects, as at least one piece of the first factor information, at least one of the outside air temperature, the weight, the exercise information, the medication information, and the salt intake.

According to the above configuration, the user can ascertain factor information that is highly relevant to blood pressure fluctuations over a unit of long-term period.

In another example of the present disclosure, the plurality of units of period include a unit of a year, a unit of a month, a unit of a week, and a unit of a day. When the designated unit of period is the unit of a week or the unit of a day, the selection unit selects, as at least one piece of the first factor information, at least one of the room temperature, the sleep information, the stress information, the medication information, and the smoking information.

According to the above configuration, the user can ascertain factor information that is highly relevant to blood pressure fluctuations over a unit of short-term period.

### Advantageous Effects of Invention

According to the present disclosure, the user convenience can be improved by presenting the relevance between blood pressure fluctuations during a designated period and factors that affect blood pressure fluctuations during that period.

### BRIEF DESCRIPTION OF DRAWINGS

Various embodiments are disclosed, by way of example only, with reference to the accompanying schematic drawings in which corresponding reference symbols indicate corresponding parts, in which:

FIG. 1 is a diagram illustrating an information processing system according to an embodiment;

FIG. 2 is a block diagram illustrating an example of a hardware configuration of a blood pressure monitor;

FIG. 3 is a block diagram illustrating an example of a hardware configuration of a terminal device;

FIG. 4 is a block diagram illustrating an example of a functional configuration of the terminal device;

FIG. 5 is a diagram illustrating an example of a display screen of the terminal device;

FIG. 6 is a diagram illustrating another example of the display screen of the terminal device;

FIG. 7 is a diagram illustrating still another example of the display screen of the terminal device;

FIG. 8 is a diagram for describing an example of a factor information priority display method;

FIG. 9 is a diagram for describing another example of the factor information priority display method;

FIG. 10 is a flowchart illustrating an example of a processing procedure of the blood pressure monitor; and,

FIG. 11 is a diagram illustrating an example of a processing procedure of the terminal device.

### DESCRIPTION OF EMBODIMENTS

It is understood that this disclosure is not limited to the particular methodology, materials and modifications described and as such may, of course, vary. It is also understood that the terminology used herein is for the purpose of describing particular aspects only, and is not intended to limit the scope of the claims. Those in the art will understand that any suitable material, now known, or hereafter developed, may be used in forming the present invention described herein.

Unless defined otherwise, all technical and scientific terms used herein have the same meaning as commonly understood to one of ordinary skill in the art to which this disclosure pertains. It should be understood that any methods, devices or materials similar or equivalent to those described herein can be used in the practice or testing of the example embodiments.

The word “example” or “exemplary” is used herein to mean “serving as an example, instance, or illustration.” Any implementation described herein as “exemplary” is not necessarily to be construed as preferred or advantageous over other implementations.

If the specification states a component or feature “may,” “can,” “could,” “should,” “would,” “preferably,” “possibly,” “typically,” “optionally,” “for example,” “often,” or “might” (or other such language) be included or have a characteristic, that a specific component or feature is not required to be included or to have the characteristic. Such a component or feature may be optionally included in some embodiments, or it may be excluded.

Embodiments of the present invention will be described below with reference to the drawings. In the following description, the same components are denoted by the same reference numerals. Names and functions thereof are also the same. Thus, the detailed description of such components is not repeated.

### Application Example

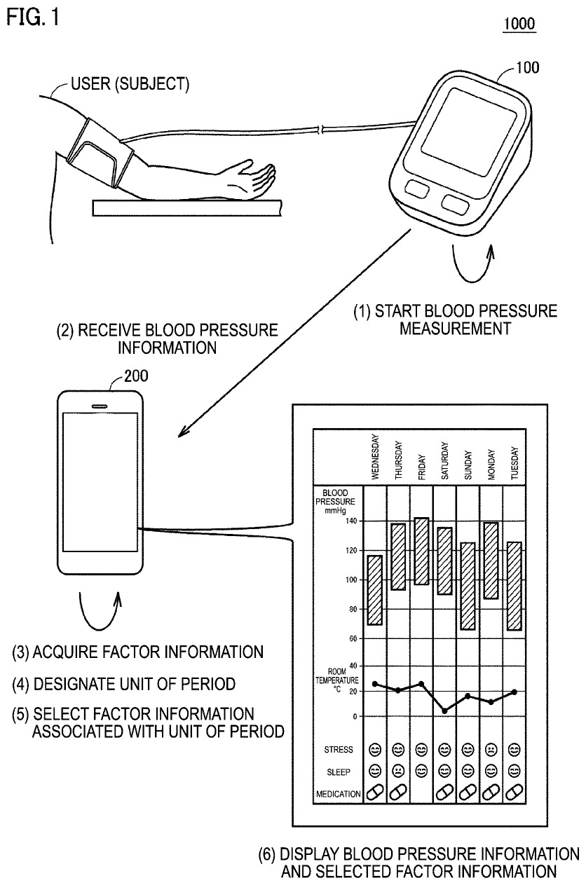

An application example of the present invention will be described. FIG. 1 is a diagram illustrating an information processing system 1000 according to the present embodiment.

Referring to FIG. 1, the information processing system 1000 includes a blood pressure monitor 100 that measures the blood pressure of a subject, and a terminal device 200 that is configured to be able to communicate with the blood pressure monitor 100. Specifically, the blood pressure monitor 100 is an upper-arm blood pressure monitor that measures the blood pressure of a user who is a subject. The blood pressure monitor 100 includes a main body and a cuff (an arm band) as main components. The blood pressure monitor 100 may be a wrist-type blood pressure monitor in which a main body and a cuff (an arm band) are integrated together. In the following, for ease of description, it is assumed that the subject (user) of the blood pressure monitor 100 is the user of the terminal device 200.

In FIG. 1, a situation is assumed in which a user measures his or her own blood pressure using the blood pressure monitor 100. Upon receiving the blood pressure measurement instruction, the blood pressure monitor 100 starts blood pressure measurement (corresponding to (1) in FIG. 1). For example, the blood pressure monitor 100 extracts a pulse wave signal (fluctuation component) superimposed on a cuff pressure indicating the internal pressure of a cuff attached to a user's part to be measured (e.g., the arm), and calculates a blood pressure value using the oscillometric method based on the pulse wave signal. The blood pressure monitor 100 stores blood pressure information such as blood pressure values in an internal memory. The blood pressure information includes the measurement date and time of the blood pressure, systolic blood pressure (maximum blood pressure), diastolic blood pressure (minimum blood pressure), and the like. The blood pressure information may be stored in association with the user's identification information (e.g., user ID).

The terminal device 200 acquires (receives) the user's blood pressure information transmitted from the blood pressure monitor 100 and stores the blood pressure information in the internal memory (corresponding to (2) in FIG. 1). The terminal device 200 activates a management application that manages biological information such as blood pressure, acquires factor information that affects blood pressure fluctuations, and stores the information in the internal memory (corresponding to (3) in FIG. 1). The factor information includes, for example, the room temperature at the time of blood pressure measurement, the outdoor temperature (outside air temperature), the user's weight, exercise information, sleep information, stress information, medication information, smoking information, and salt intake. A method for acquiring each piece of factor information will be described later. The factor information is associated with date and time information such as the measurement date and time, acquisition date and time, etc. of the factor information.

The terminal device 200 receives, from the user, a designation input of a unit for a period (hereinafter also referred to as a “unit of period”) for displaying blood pressure values and the like (corresponding to (4) in FIG. 1). The unit of period includes a unit of a day, a unit of a week, a unit of a month, or a unit of a year. Specifically, the “unit of a day” refers to one day as a unit of period, the “unit of a week” refers to one week as a unit of period, the “unit of a month” refers to one month as a unit of period, and the “unit of a year” refers to one year as a unit of period. Here, it is assumed that the unit of period designated (hereinafter also referred to as a “designated unit of period”) is a “unit of a week.”

The terminal device 200 selects one or more pieces of factor information associated with a designated unit of period (here, “unit of a week”) from among the plurality of pieces of factor information stored in the internal memory (corresponding to (5) in FIG. 1). For example, room temperature, stress information, sleep information, and medication information are associated with “unit of a week.”

The terminal device 200 displays the blood pressure information and the selected factor information on the display screen along the time axis based on the designated unit of period (corresponding to (6) in FIG. 1). In the example of FIG. 1, one week's worth of blood pressure values and a plurality of pieces of factor information (room temperature, stress information, sleep information, and medication information) are displayed in parallel along the time axis.

According to the above application example, the terminal device 200 selects factor information suitable for a unit of period designated by the user, and displays the factor information in time series together with blood pressure information. Therefore, the user can simultaneously check his or her own blood pressure fluctuations and factor information that is likely to affect the blood pressure fluctuations, and can thus clearly recognize the relevance between blood pressure fluctuations and factor information.

Furthermore, the user can simply designate a desired unit of period, and factor information highly relevant to blood pressure fluctuations during that period will be automatically displayed, allowing the user to easily and appropriately manage their own blood pressure. Furthermore, since factor information that is less relevant to blood pressure fluctuations in that unit of period is not displayed, the user will not be misled.

### Configuration Example—Hardware Configuration

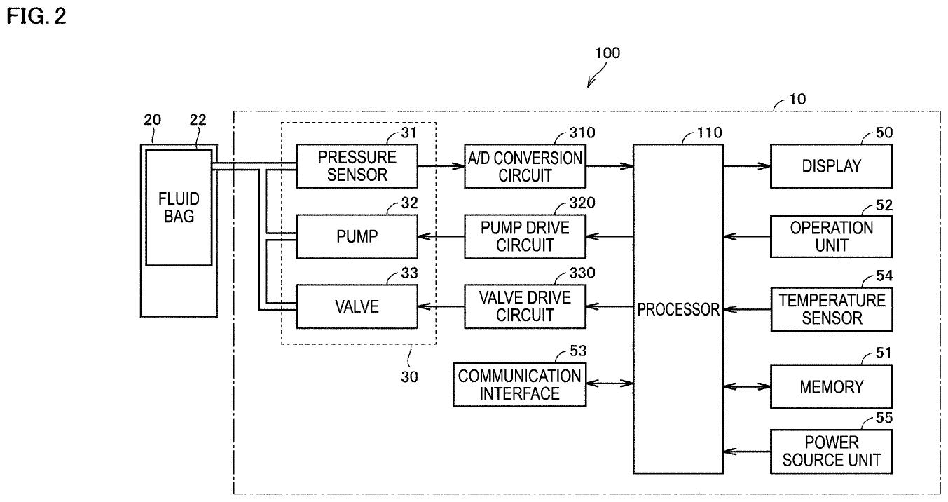

FIG. 2 is a block diagram illustrating an example of a hardware configuration of the blood pressure monitor 100. Referring to FIG. 2, the blood pressure monitor 100 includes a main body 10 and a cuff 20 as main components. A fluid bag 22 is interiorly contained in the cuff 20. The main body 10 includes a processor 110, an air-system component 30 for blood pressure measurement, an A/D conversion circuit 310, a pump drive circuit 320, a valve drive circuit 330, a display 50, a memory 51, an operation unit 52, a communication interface 53, a temperature sensor 54, and a power source unit 55.

The processor 110 is an arithmetic processing unit such as a central processing unit (CPU) or a multi-processing unit (MPU). The processor 110 reads and executes a program stored in the memory 51 and thereby implements each of processes (steps) of the blood pressure monitor 100 described below. For example, the processor 110 performs control of driving a pump 32 and a valve 33 in accordance with an operation signal from the operation unit 52. In addition, the processor 110 calculates a blood pressure value by using an algorithm for blood pressure calculation according to the oscillometric method and displays the blood pressure value on the display 50.

The memory 51 is achieved by a random access memory (RAM), a read-only memory (ROM), a flash memory, or the like. The memory 51 stores a program for controlling the blood pressure monitor 100, data used to control the blood pressure monitor 100, setting data for setting various functions of the blood pressure monitor 100, data of measurement results of blood pressure values, pulse rates, pulse wave signals, and the like. The memory 51 is also used as a work memory when a program is executed.

The air-system component 30 supplies or discharges air through an air line to or from the fluid bag 22 interiorly contained in the cuff 20. The air-system component 30 includes a pressure sensor 31 for detecting pressure inside the fluid bag 22, and the pump 32 and the valve 33 that serve as an expanding/contracting mechanism section for expanding/contracting the fluid bag 22.

The pressure sensor 31 detects the pressure (cuff pressure) inside the fluid bag 22 and outputs a signal (cuff pressure signal) corresponding to the detected pressure to the A/D conversion circuit 310. The pressure sensor 31 is, for example, a piezoresistive pressure sensor, and is connected to the pump 32, the valve 33, and the fluid bag 22 interiorly contained in the cuff 20 via the air line. The pump 32 supplies air as a fluid to the fluid bag 22 through the air line in order to increase the cuff pressure. The valve 33 is opened and closed to control the cuff pressure by discharging air inside the fluid bag 22 through the air line or filling air into the fluid bag 22.

The A/D conversion circuit 310 converts an output value of the pressure sensor 31 (e.g., a voltage value corresponding to a change in electric resistance due to a piezoresistive effect) from an analog signal to a digital signal and outputs the converted signal to the processor 110. The processor 110 acquires a signal representing the cuff pressure in accordance with the output value of the A/D conversion circuit 310. The pump drive circuit 320 controls driving of the pump 32 based on a control signal provided from the processor 110. The valve drive circuit 330 controls opening and closing of the valve 33 based on a control signal provided from the processor 110.

The display 50 displays various types of information including a blood pressure measurement result and the like based on a control signal from the processor 110. The various types of information includes the current date and time, systolic blood pressure, diastolic blood pressure, pulse rate, room temperature, and the like. The operation unit 52 inputs an operation signal corresponding to an instruction from the subject to the processor 110. The operation unit 52 includes, for example, a measurement switch for receiving a blood pressure measurement instruction. The communication interface 53 exchanges various types of information with an external device (e.g., the terminal device 200). As a communication method, for example, a wireless communication method such as Bluetooth (registered trademark) low energy (BLE) or wireless local area network (LAN) is employed. The temperature sensor 54 measures the ambient temperature (room temperature). The power source unit 55 supplies power to the processor 110 and each piece of hardware.

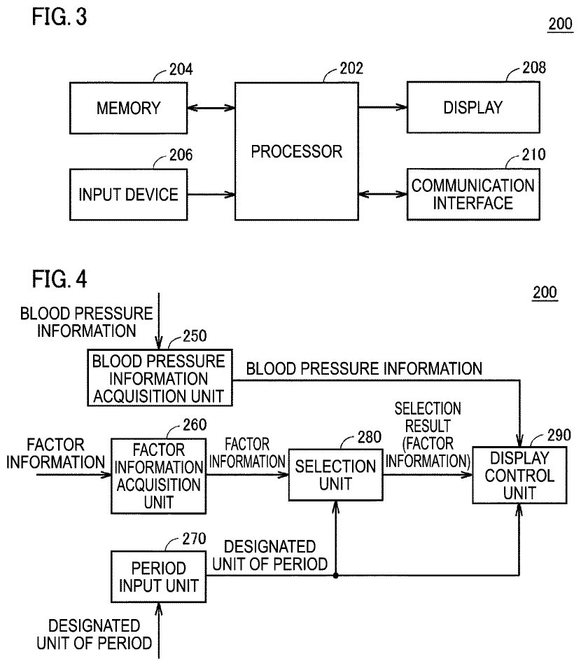

FIG. 3 is a block diagram illustrating an example of a hardware configuration of the terminal device 200. Referring to FIG. 3, the terminal device 200 includes, as main components, a processor 202, a memory 204, an input device 206, a display 208, and a communication interface 210.

The processor 202 reads and executes a program stored in the memory 204 to implement each of the processes (steps) of the terminal device 200 described below. The memory 204 is implemented by a RAM, a ROM, a flash memory, or the like. The input device 206 receives operation input for the terminal device 200. Typically, the input device 206 is implemented by a button, a touch panel provided on the display 208, or the like. The communication interface 210 is a communication interface for exchanging various types of data between the terminal device 200 and the blood pressure monitor 100.

### Configuration Example—Functional Configuration

FIG. 4 is a block diagram illustrating an example of a functional configuration of the terminal device 200. Referring to FIG. 4, the terminal device 200 includes, as main functional configurations, a blood pressure information acquisition unit 250, a factor information acquisition unit 260, a period input unit 270, a selection unit 280, and a display control unit 290. Each of these functions is implemented, for example, by the processor 202 of the terminal device 200 executing a program stored in the memory 204. Note that some or all of these functions may be configured to be implemented by hardware.

The blood pressure information acquisition unit 250 acquires (receives) blood pressure information on the subject (user) measured using the blood pressure monitor 100. The blood pressure information acquisition unit 250 receives the blood pressure information transmitted from the blood pressure monitor 100 by requesting the blood pressure information from the blood pressure monitor 100. Alternatively, the blood pressure information acquisition unit 250 determines the timing for transmission by the blood pressure monitor 100, and receives the blood pressure information transmitted from the blood pressure monitor 100 at the point in time when the timing is reached.

The factor information acquisition unit 260 acquires a plurality of pieces of factor information that affect the blood pressure fluctuation of the subject. The plurality of pieces of factor information include the room temperature and outside air temperature at the time of blood pressure measurement, a weight, exercise information, sleep information, stress information, medication information, smoking information, and salt intake of the subject. An example of the method for acquiring each piece of factor information will be described.

The factor information acquisition unit 260 acquires (receives) the room temperature measured using the blood pressure monitor 100 and the measurement date and time from the blood pressure monitor 100. The factor information acquisition unit 260 periodically acquires (receives) the outside air temperature from a server that provides weather information. The acquired outside air temperature is typically the outside air temperature in the area where the subject lives.

The factor information acquisition unit 260 acquires (receives) the weight of the subject from a weighing scale with a communication function that has measured the weight of the subject. The measurement data of the weighing scale includes weight (measurement value) and measurement date and time. The factor information acquisition unit 260 acquires (receives) the number of steps measured using a predetermined pedometer as exercise information, for example. The measurement data of the pedometer includes the number of steps (measurement value) and the measurement date and time. The factor information acquisition unit 260 may acquire, as exercise information, the number of steps measured using a step counting application installed on the terminal device 200.

The factor information acquisition unit 260 acquires (receives) information relating to sleep (sleep information) measured using a predetermined sleep measurement device. The sleep measurement device is a wearable device that is worn on the user's body, such as a wristwatch, a ring, or an eye mask. The sleep information includes an evaluation result obtained by evaluating the user's sleep quality (for example, “good,” “poor,” etc.) and the measurement date and time. The factor information acquisition unit 260 may acquire sleep information measured by a sleep measurement application installed on the terminal device 200.

The factor information acquisition unit 260 receives input of the user's stress information, medication information, and smoking information via the input device 206. The medication information includes whether medication has been taken and the date and time when the medication was taken. The stress information includes whether there is stress and the date and time when the input of the stress information was received. The smoking information includes whether there is smoking and the date and time of smoking.

The factor information acquisition unit 260 receives input of the amount of salt ingested by the user via the input device 206. The amount of salt is the salt amount of the user's diet measured using a predetermined salt meter. Furthermore, the factor information acquisition unit 260 may acquire the amount of salt calculated by a known dietary management application installed on the terminal device 200. The dietary management application calculates the amount of salt by analyzing the dietary details input by the user. The factor information acquisition unit 260 calculates the user's daily salt intake based on the acquired amount of salt.

The factor information acquisition unit 260 is only required to be able to acquire each piece of factor information described above, and the acquisition method is not limited to the above-mentioned acquisition method.

The period input unit 270 receives input of a designated unit of period designated by the user via the input device 206. Specifically, the user uses the input device 206 to designate a desired unit of period (for example, a unit of a week) from among a plurality of units of period (for example, a unit of a day, a unit of a week, a unit of a month, and a unit of a year).

The selection unit 280 selects factor information X associated with the designated unit of period from among a plurality of pieces of factor information. When the designated unit of period is set to a unit of a year or a unit of a month, the selection unit 280 selects, as the plurality of pieces of factor information X, outside air temperature, weight, exercise information, medication information, and salt intake. The selection unit 280 may select at least one piece of factor information X (that is, at least one of the outside air temperature, weight, exercise information, medication information, and salt intake).

Furthermore, when the designated unit of period is a unit of a week or a unit of a day, the selection unit 280 selects, as the plurality of pieces of factor information X, room temperature, sleep information, stress information, medication information, and smoking information. The selection unit 280 may select at least one piece of factor information X (that is, at least one of room temperature, sleep information, stress information, medication information, and smoking information).

Factor information that is considered to affect long-term blood pressure fluctuations is associated with a unit of long-term period (e.g., a unit of a year or a unit of a month), and factor information that is considered to affect short-term blood pressure fluctuations is associated with a unit of short-term period (e.g., a unit of a week or a unit of a day). The units of period associated with each piece of factor information will be specifically described below.

When the outside air temperature is low, such as in winter, blood vessels constrict, and blood pressure tends to rise, while when the outside air temperature is high, such as in summer, blood vessels expand, and blood pressure tends to fall. Generally, blood pressure is measured in a room, and therefore it is unlikely that outside air temperature will have an immediate effect on blood pressure. Therefore, outside air temperature is associated with units of long-term period.

It is known that increased weight increases blood pressure, and decreased weight decreases blood pressure. Generally, weight does not fluctuate suddenly, but rather fluctuates gradually over the long term. Therefore, weight is associated with units of long-term period.

Continuous aerobic exercise (e.g., walking) is known to lower blood pressure. Therefore, exercise information (e.g., number of steps) is associated with units of long-term period.

It is known that increased salt intake increases blood pressure, and decreased salt intake decreases blood pressure. For example, in the treatment of high blood pressure, measures such as limiting the salt intake per day are taken. In general, reducing blood pressure is expected by continuing to limit salt intake over the long term. Therefore, salt intake is associated with units of long-term period.

Generally, blood pressure is measured in a room, and therefore the temperature of the room where the blood pressure is measured (that is, room temperature) immediately affects the blood pressure. Therefore, room temperature is associated with units of short-term period.

It is known that when sleep time is short and the quality of sleep is poor, the sympathetic nervous system becomes activated, and blood pressure rises. Therefore, sleep information is associated with units of short-term period. Blood pressure can rise sharply due to physical or mental stress. Therefore, stress information is associated with units of short-term period. It is known that smoking has the effect of constricting blood vessels, and therefore blood pressure rises after smoking. Therefore, smoking information is associated with units of short-term period.

To treat high blood pressure, antihypertensive drugs may be taken to lower blood pressure. Typically, antihypertensive drugs are prescribed to lower blood pressure gradually, rather than suddenly. Therefore, medication information is associated with units of long-term period. On the other hand, the user may want to ascertain how blood pressure changed between days when antihypertensive drugs were taken and days when antihypertensive drugs were not taken. Therefore, medication information is also associated with units of short-term period.

The display control unit 290 displays the blood pressure information and the factor information X on the display screen along the time axis based on the designated unit of period. For example, when the designated unit of period is a unit of a week, the display control unit 290 displays blood pressure information and factor information X selected by the selection unit 280 (e.g., room temperature, sleep information, stress information, medication information, and smoking information) on the display screen along the time axis.

In another aspect, the selection unit 280 selects factor information Y that is not associated with the designated unit of period. The display control unit 290 displays the blood pressure information, the factor information X, and the factor information Y on the display screen along the time axis based on the designated unit of period. In this case, the blood pressure information and the factor information X are each displayed with priority over the factor information Y.

Specifically, the display control unit 290 displays each of the blood pressure information and the factor information X with priority over the factor information Y by displaying the factor information Y in a grayed-out manner. Furthermore, the display control unit 290 controls a display order of the blood pressure information, the factor information X, and the factor information Y such that the blood pressure information and the factor information X are each displayed with priority over the factor information Y. Specific display examples by the display control unit 290 will be described later.

### Configuration Example—Display Examples

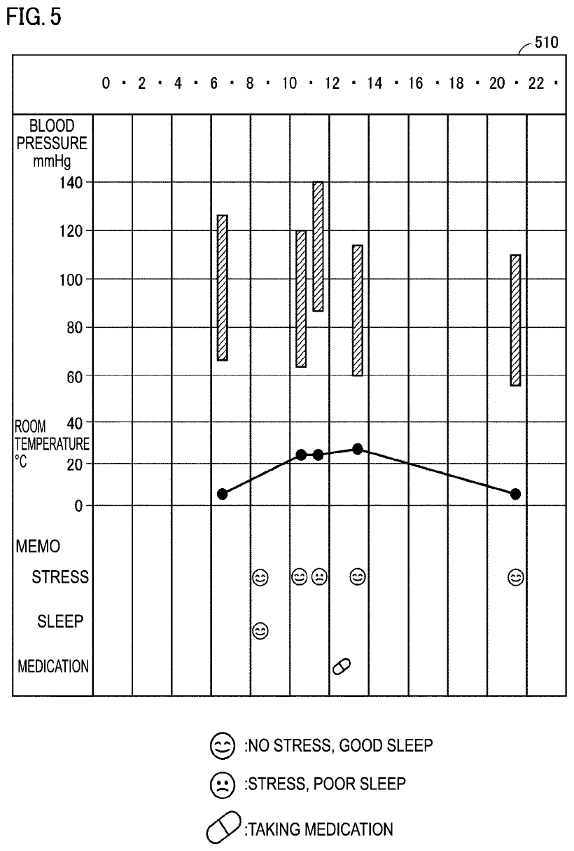

FIG. 5 is a diagram illustrating an example of a display screen of the terminal device 200. Referring to FIG. 5, a display screen 510 is an example of a screen that is displayed on the display 208 when the designated unit of period is a unit of a day. The display screen 510 displays blood pressure information and factor information X (here, room temperature, stress information, sleep information, and medication information) associated with the unit of a day in time series. Specifically, the blood pressure information and the factor information X for one day (24 hours) are displayed in parallel along the time axis (for example, every hour).

The display screen 510 includes a bar graph showing blood pressure values, a line graph showing the room temperature at the time of blood pressure measurement, an object showing whether there is stress, an object showing the quality of sleep (good or poor), and an object showing whether medication is taken. The top and bottom of the bar graph showing the blood pressure values indicate the systolic blood pressure and the diastolic blood pressure, respectively.

The display screen 510 shows that at around 8 o'clock, there is no stress, and the sleep state is good. This suggests that stress may have caused blood pressure to rise around 11 o'clock. It also shows that the medicine was taken around 12 o'clock.

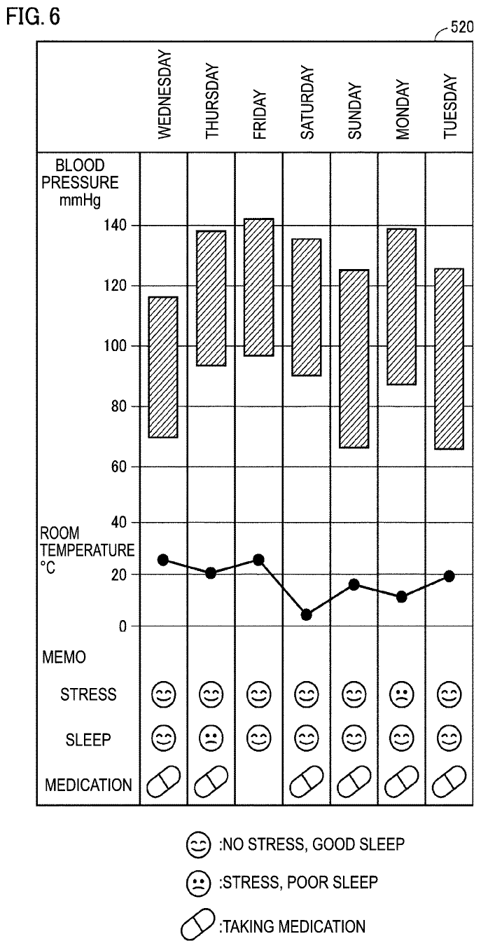

FIG. 6 is a diagram illustrating another example of the display screen of the terminal device 200. Referring to FIG. 6, a display screen 520 is an example of a screen that is displayed on the display 208 when the designated unit of period is a unit of a week. The display screen 520 displays one week's worth of blood pressure information and factor information X (here, room temperature, stress information, sleep information, and medication information) associated with the unit of a week in time series. Specifically, the blood pressure information and the factor information X for one week (7 days) are displayed in parallel along the time axis (for example, every day).

Since the same factor information X is associated with the unit of a day and the unit of a week, the display content of the display screen 520 is similar to the display content of the display screen 510. The blood pressure value for a certain day (for example, 8th) indicates the average value of the blood pressure measured on the 8th. The top and bottom of the bar graph indicate the average systolic and diastolic blood pressure values for the day, respectively. The room temperature indicates the average value of the room temperature at the time of blood pressure measurement during the day. The stress information indicates whether there is stress throughout the day. The sleep information indicates the quality of sleep (good or poor) for that day. The medication information indicates whether medication was taken on that day.

The display screen 520 suggests that the blood pressure is relatively high on the 9th due to lack of sleep, on the 11th due to a drop in room temperature, and on the 13th due to stress.

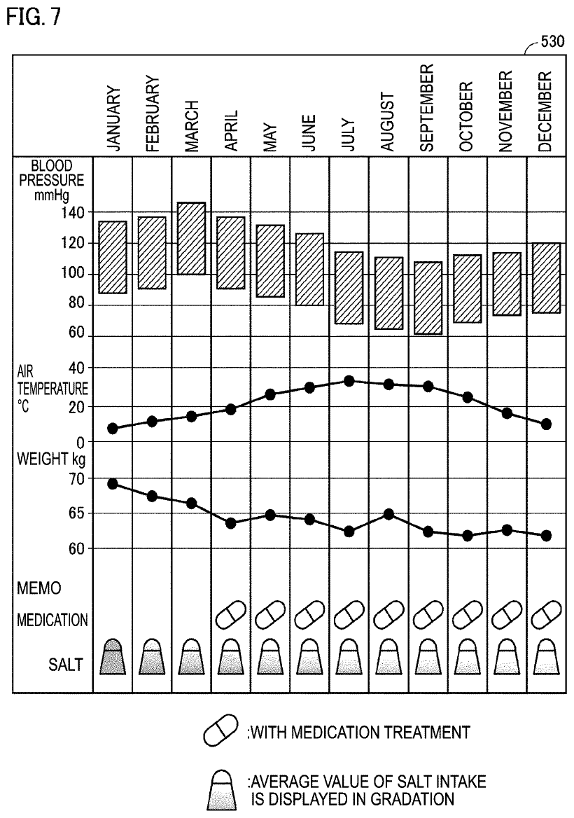

FIG. 7 is a diagram illustrating still another example of the display screen of the terminal device 200. Referring to FIG. 7, a display screen 530 is an example of a screen that is displayed on the display 208 when the designated unit of period is a unit of a year. The display screen 530 displays one year's worth of blood pressure information and factor information X (here, temperature, weight, medication information, and salt intake) associated with the unit of a year in time series. Specifically, the blood pressure information and the factor information X for one year (12 months) are displayed in parallel along the time axis (for example, every month).

The display screen 530 includes a bar graph showing an average blood pressure value for the month, a line graph showing an average temperature (outside air temperature) for the month, a line graph showing an average weight for the month, an object showing whether medication is taken for the month, and an object showing an average value of salt intake for the month. The top and bottom of the bar graph indicate the average systolic and diastolic blood pressure values for the month, respectively. The object for salt intake is displayed darker the higher the average value of salt intake.

The display screen 530 suggests that blood pressure is on a downward trend due to weight loss, medication treatment, and reduced salt intake, in accordance with blood pressure fluctuations caused by changes in outside air temperature.

The same applies to the display screen displayed on the display 208 when the designated unit of period is a unit of a month. On the display screen, one month's blood pressure information and factor information X associated with the unit of a month are displayed in parallel along the time axis (for example, every week).

The display screen 510 of FIG. 5 and the display screen 520 of FIG. 6 described above only display factor information X (e.g., room temperature, stress information, sleep information, and medication information) associated with a unit of short-term period, and do not display factor information Y that is not associated with the unit of short-term period. Similarly, the display screen 530 in FIG. 7 displays only factor information X (e.g., outside air temperature, weight, medication information, and salt intake) associated with a unit of long-term period, and does not display factor information Y that is not associated with the unit of long-term period.

However, as will be described below, the terminal device 200 may employ a display method in which factor information X is displayed with priority over factor information Y.

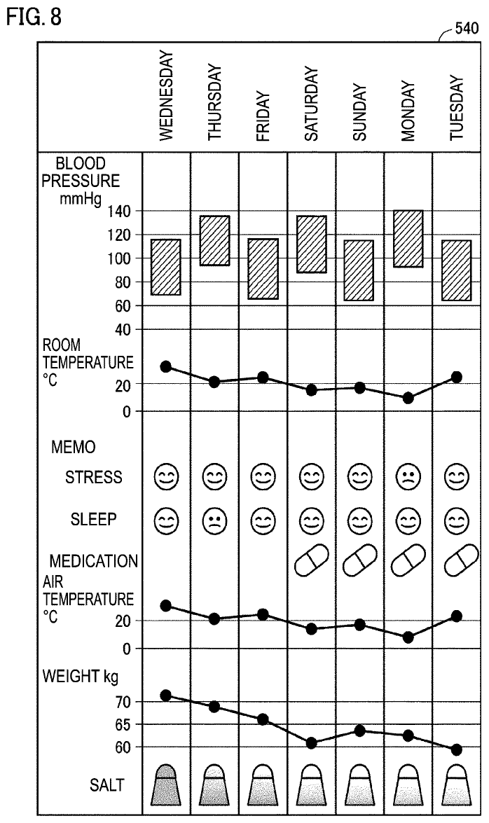

FIG. 8 is a diagram for describing an example of a factor information priority display method. Referring to FIG. 8, a display screen 540 is a screen that is displayed on the display 208 when the designated unit of period is a unit of a week. In addition to the blood pressure information and the factor information X (here, room temperature, stress information, sleep information, and medication information) that were displayed on the display screen 520 in the unit of a week illustrated in FIG. 6, the display screen 540 also displays the factor information Y (here, outside air temperature, body weight, and salt intake).

Here, the blood pressure information and the factor information X are displayed in the upper part of the screen, while the factor information Y is displayed in the lower part of the screen. Specifically, the processor 202 (display control unit 290) controls the display order such that the blood pressure information and the factor information X are each displayed with priority over the factor information Y.

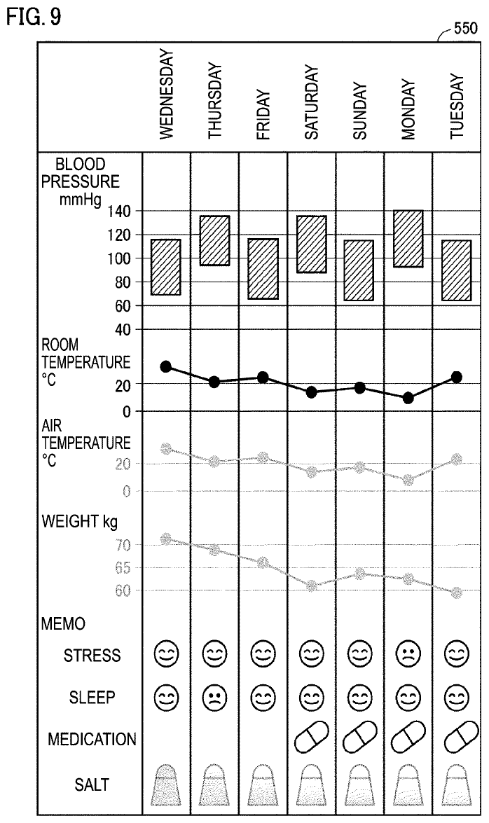

FIG. 9 is a diagram for describing another example of the factor information priority display method. Referring to FIG. 9, a display screen 550 is a screen that is displayed on the display 208 when the designated unit of period is a unit of a week. The display screen 550 displays the same items as those in FIG. 8 (that is, blood pressure information, factor information X, and factor information Y).

However, the factor information Y is displayed in a grayed-out manner, making it more difficult to visually recognize than the blood pressure information and the factor information X. In this way, the processor 202 (display control unit 290) displays each of the blood pressure information and the factor information X with priority over the factor information Y by displaying the factor information Y in a grayed-out manner.

According to the display screen 540 and the display screen 550, the factor information X having high relevance in the designated unit of period is displayed with priority over the factor information Y having low relevance. Therefore, the user can easily check the factor information X that requires attention, and can also check the factor information Y as reference information.

### Processing Procedure

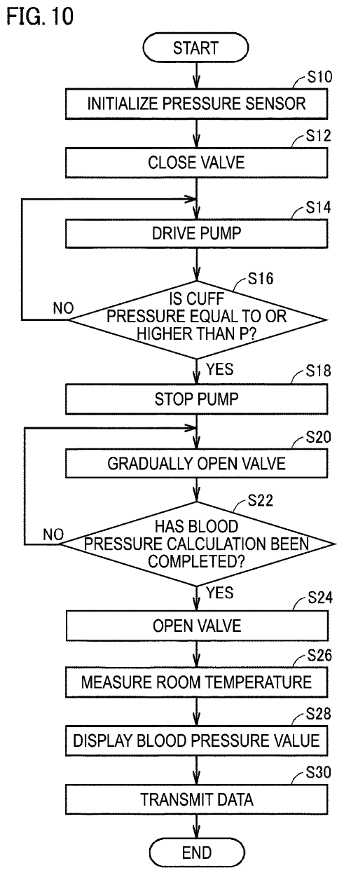

FIG. 10 is a flowchart illustrating an example of a processing procedure of the blood pressure monitor 100. Here, it is assumed that blood pressure measurement is executed using a reduced pressure measurement method, but blood pressure measurement may also be executed using a pressurized measurement method.

Referring to FIG. 10, the processor 110 of the blood pressure monitor 100 initializes the pressure sensor 31 (step S10). Specifically, the processor 110 initializes the processing memory area, and performs 0 mmHg adjustment (setting the atmospheric pressure to 0 mmHg) of the pressure sensor 31 in a state where the pump 32 is turned off (stopped) and the valve 33 is open.

The processor 110 closes the valve 33 via the valve drive circuit 330 (step S12), and drives the pump 32 via the pump drive circuit 320 to start pressurization of the cuff 20 (fluid bag 22) (step S14). At this time, the processor 110 controls the pressurization rate of the cuff pressure that is pressure inside the fluid bag 22 based on the output of the pressure sensor 31 while supplying air from the pump 32 through the air line to the fluid bag 22.

The processor 110 determines whether the cuff pressure reaches or exceeds a threshold value P (step S16). Typically, the threshold value P is set to a value 30 mmHg to 40 mmHg higher than the expected maximum blood pressure (systolic blood pressure).

When the cuff pressure is lower than the threshold value P (NO in step S16), the processor 110 returns to step S14. When the cuff pressure is equal to or greater than the threshold value P (YES in step S16), the processor 110 stops the pump 32 (step S18) and controls the valve 33 to gradually open (step S20). Accordingly, the cuff pressure gradually decreases from the pressurization process to the depressurization process.

In the depressurization process, the processor 110 extracts a pulse wave signal from the cuff pressure signal detected by the pressure sensor 31, attempts to calculate systolic blood pressure and diastolic blood pressure based on the pulse wave signal, and determines whether the blood pressure calculation has been completed (step S22). When the blood pressure calculation has not been completed (NO in step S22), the processor 110 repeats the processing of steps S20 and S22. When the blood pressure calculation has been completed (YES in step S22), the processor 110 performs control to fully open the valve 33 (step S24) and rapidly discharge the air inside the cuff 20.

The processor 110 measures the room temperature via the temperature sensor 54 (step S26), and displays the blood pressure value and other data measured in the blood pressure measurement on the display 50 (step S28). The processor 110 transmits data such as blood pressure information and room temperature to the terminal device 200 via the communication interface 53 (step S30). Data such as blood pressure information and room temperature is stored in the memory 51 of the blood pressure monitor 100.

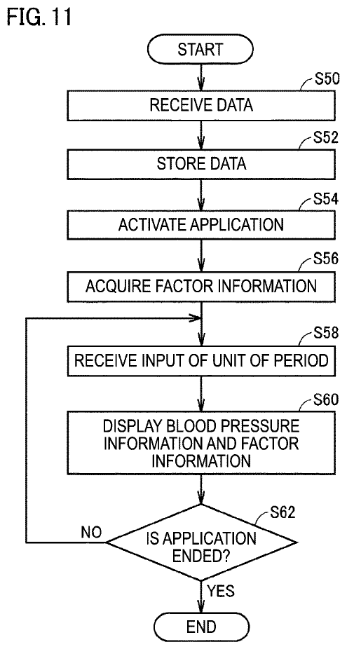

FIG. 11 is a diagram illustrating an example of a processing procedure of the terminal device 200. Referring to FIG. 11, the processor 202 of the terminal device 200 receives data such as blood pressure information and room temperature via the communication interface 210 (step S50). The processor 202 stores the data in the memory 204 (step S52). The processor 202 receives an input to activate the management application via the input device 206, and activates the management application (step S54). The processor 202 acquires various types of factor information using the acquisition method described above (step S56).

The processor 202 receives a designation input of a unit of period via the input device 206 (step S58). The processor 202 displays the blood pressure information and the factor information on the display 208 along the time axis based on the designated unit of period (step S60). For example, the processor 202 displays the display screens 510 to 550 on the display 208. The processor 202 determines whether an instruction to end the management application has been received via the input device 206 (step S62). When the end instruction has not been received (NO in step S62), processor 202 returns to step S58. When the end instruction has been received (YES in step S62), the processor 202 ends the process. For example, the processor 202 transitions into background operation and executes steps S50, S52, and the like.

### Other Embodiments

(1) In the embodiments described above, a program may be provided that causes a computer to function and execute controls such as those described in the aforementioned flowcharts. Such a program can also be provided as a program product by being recorded on a non-transitory computer-readable recording medium such as a flexible disk, a secondary storage device, a main storage device, or a memory card attached to a computer. Alternatively, a program may be provided by recording the program on a recording medium such as a hard disk built into a computer. The program can also be provided by downloading it over a network.

(2) The configuration exemplified as the embodiments described above is an example of a configuration of the present invention, and the configuration can be combined with other known technology, and a part of the configuration may be omitted or modified without departing from the scope of the present invention. In addition, the processes and configurations described in other embodiments may be employed as appropriate in the embodiments described above.

### Supplementary Notes

As described above, the present embodiment includes the following disclosures.

Configuration 1: A terminal device (200) including: a blood pressure information acquisition unit (250) configured to acquire blood pressure information on a subject measured using a blood pressure monitor (100); a factor information acquisition unit (260) configured to acquire a plurality of pieces of factor information that affect a blood pressure fluctuation of the subject; an input unit (270) configured to receive an input of a designated unit of period designated from among a plurality of units of period; a selection unit (280) configured to select, from among the plurality of pieces of factor information, first factor information associated with the designated unit of period; and a display control unit (290) configured to display the blood pressure information and the first factor information on a display screen along a time axis based on the designated unit of period.

Configuration 2: The terminal device according to configuration 1, in which the selection unit selects second factor information that is not associated with the designated unit of period, the display control unit displays the blood pressure information, the first factor information, and the second factor information on the display screen along a time axis based on the designated unit of period, and each of the blood pressure information and the first factor information is displayed with priority over the second factor information.

Configuration 3: The terminal device according to configuration 2, in which the display control unit displays each of the blood pressure information and the first factor information with priority over the second factor information by displaying the second factor information in a grayed-out manner.

Configuration 4: The terminal device according to configuration 2, in which the display control unit controls a display order of the blood pressure information, the first factor information, and the second factor information such that each of the blood pressure information and the first factor information is displayed with priority over the second factor information.

Configuration 5: The terminal device according to any one of configurations 1 to 4, in which the plurality of pieces of factor information include a room temperature and an outside air temperature at the time of blood pressure measurement, and a weight, exercise information, sleep information, stress information, medication information, smoking information, and salt intake of the subject.

Configuration 6: The terminal device according to configuration 5, in which the plurality of units of period include a unit of a year, a unit of a month, a unit of a week, and a unit of a day, and when the designated unit of period is the unit of a year or the unit of a month, the selection unit selects, as at least one piece of the first factor information, at least one of the outside air temperature, the weight, the exercise information, the medication information, and the salt intake.

Configuration 7: The terminal device according to configuration 5, in which the plurality of units of period include a unit of a year, a unit of a month, a unit of a week, and a unit of a day, and when the designated unit of period is the unit of a week or the unit of a day, the selection unit selects, as at least one piece of the first factor information, at least one of the room temperature, the sleep information, the stress information, the medication information, and the smoking information.

The embodiments disclosed herein are illustrative in all respects and are not intended as limitations. The scope of the present invention is indicated not by the descriptions above but by the claims and is intended to include all changes within the meaning and scope equal to the scope of the claims.

### REFERENCE SIGNS LIST

10 . . . Main body
20 . . . Cuff
22 . . . Fluid bag
30 . . . Air-system component
31 . . . Pressure sensor
32 . . . Pump
33 . . . Valve
50, 208 . . . Display
51, 204 . . . Memory
52 . . . Operation unit
53, 210 . . . Communication interface
54 . . . Temperature sensor
55 . . . Power source unit
100 . . . Blood pressure monitor
110, 202 . . . Processor
200 . . . Terminal device
206 . . . Input device
250 . . . Blood pressure information acquisition unit
260 . . . Factor information acquisition unit
270 . . . Period input unit
280 . . . Selection unit
290 . . . Display control unit
310 . . . A/D conversion circuit
320 . . . Pump drive circuit
330 . . . Valve drive circuit
510 to 550 . . . Display screen
1000 . . . Information processing system

What is claimed is:

1. A terminal device comprising:
a blood pressure information acquisition unit configured to acquire blood pressure information on a subject measured using a blood pressure monitor;
a factor information acquisition unit configured to acquire a plurality of pieces of factor information that affect a blood pressure fluctuation of the subject;
an input unit configured to receive an input of a designated unit of period designated from among a plurality of units of period;
a selection unit configured to select, from among the plurality of pieces of factor information, first factor information associated with the designated unit of period; and,
a display control unit configured to display the blood pressure information and the first factor information on a display screen along a time axis based on the designated unit of period.

2. The terminal device according to claim 1,
wherein the selection unit selects second factor information that is not associated with the designated unit of period,
the display control unit displays the blood pressure information, the first factor information, and the second factor information on the display screen along a time axis based on the designated unit of period, and
each of the blood pressure information and the first factor information is displayed with priority over the second factor information.

3. The terminal device according to claim 2, wherein the display control unit displays each of the blood pressure information and the first factor information with priority over the second factor information by displaying the second factor information in a grayed-out manner.

4. The terminal device according to claim 2, wherein the display control unit controls a display order of the blood pressure information, the first factor information, and the second factor information such that each of the blood pressure information and the first factor information is displayed with priority over the second factor information.

5. The terminal device according to claim 1, wherein the plurality of pieces of factor information include a room temperature and an outside air temperature at a time of blood pressure measurement, and a weight, exercise information, sleep information, stress information, medication information, smoking information, and salt intake of the subject.

6. The terminal device according to claim 2, wherein the plurality of pieces of factor information include a room temperature and an outside air temperature at a time of blood pressure measurement, and a weight, exercise information, sleep information, stress information, medication information, smoking information, and salt intake of the subject.

7. The terminal device according to claim 3, wherein the plurality of pieces of factor information include a room temperature and an outside air temperature at a time of blood pressure measurement, and a weight, exercise information, sleep information, stress information, medication information, smoking information, and salt intake of the subject.

8. The terminal device according to claim 4, wherein the plurality of pieces of factor information include a room temperature and an outside air temperature at a time of blood pressure measurement, and a weight, exercise information, sleep information, stress information, medication information, smoking information, and salt intake of the subject.

9. The terminal device according to claim 5, wherein:
the plurality of units of period include a unit of a year, a unit of a month, a unit of a week, and a unit of a day; and,
when the designated unit of period is the unit of a year or the unit of a month, the selection unit selects, as at least one piece of the first factor information, at least one of the outside air temperature, the weight, the exercise information, the medication information, and the salt intake.

10. The terminal device according to claim 6, wherein:
the plurality of units of period include a unit of a year, a unit of a month, a unit of a week, and a unit of a day; and,
when the designated unit of period is the unit of a year or the unit of a month, the selection unit selects, as at least one piece of the first factor information, at least one of the outside air temperature, the weight, the exercise information, the medication information, and the salt intake.

11. The terminal device according to claim 7, wherein:
the plurality of units of period include a unit of a year, a unit of a month, a unit of a week, and a unit of a day; and,
when the designated unit of period is the unit of a year or the unit of a month, the selection unit selects, as at least one piece of the first factor information, at least one of the outside air temperature, the weight, the exercise information, the medication information, and the salt intake.

12. The terminal device according to claim 8, wherein:
the plurality of units of period include a unit of a year, a unit of a month, a unit of a week, and a unit of a day; and,
when the designated unit of period is the unit of a year or the unit of a month, the selection unit selects, as at least one piece of the first factor information, at least one of the outside air temperature, the weight, the exercise information, the medication information, and the salt intake.

13. The terminal device according to claim 5, wherein:
the plurality of units of period include a unit of a year, a unit of a month, a unit of a week, and a unit of a day; and,
when the designated unit of period is the unit of a week or the unit of a day, the selection unit selects, as at least one piece of the first factor information, at least one of the room temperature, the sleep information, the stress information, the medication information, and the smoking information.

14. The terminal device according to claim 6, wherein:
the plurality of units of period include a unit of a year, a unit of a month, a unit of a week, and a unit of a day; and,
when the designated unit of period is the unit of a week or the unit of a day, the selection unit selects, as at least one piece of the first factor information, at least one of the room temperature, the sleep information, the stress information, the medication information, and the smoking information.

15. The terminal device according to claim 7, wherein:
the plurality of units of period include a unit of a year, a unit of a month, a unit of a week, and a unit of a day; and,
when the designated unit of period is the unit of a week or the unit of a day, the selection unit selects, as at least one piece of the first factor information, at least one of the room temperature, the sleep information, the stress information, the medication information, and the smoking information.

16. The terminal device according to claim 8, wherein:
the plurality of units of period include a unit of a year, a unit of a month, a unit of a week, and a unit of a day; and,
when the designated unit of period is the unit of a week or the unit of a day, the selection unit selects, as at least one piece of the first factor information, at least one of the room temperature, the sleep information, the stress information, the medication information, and the smoking information.
